(19) United States
(12) Patent Application Publication
(10) Pub. No.: US 2012/0156765 A1
(43) Pub. Date: Jun. 21, 2012

(54) FLUID FILTERING DEVICE

(75) Inventors: Reinhold Wimberger-Friedl, Veldhoven (NL); Jacobus Hermanus Maria Neijzen, Heeze (NL); Joost Hubert Maas, Helmond (NL)

(73) Assignee: KONINKLIJKE PHILIPS ELECTRONICS N.V., EINDHOVEN (NL)

(21) Appl. No.: 13/393,097

(22) PCT Filed: Aug. 25, 2010
(86) PCT No.: PCT/IB2010/053811

§ 371 Date: Feb. 28, 2012

(30) Foreign Application Priority Data
Sep. 1, 2009 (EP) 09169137.8

(51) Int. Cl. C12M 1/34; G01N 30/00; B01D 37/00; B23P 17/00; B01D 35/00; B01D 35/01

(52) U.S. Cl. 435/287.1; 210/483; 210/472; 210/767; 29/527.1; 422/69

(57) ABSTRACT

The invention relates to a fluid filtering device comprising a filter (2) for filtering a fluid and a substrate (3) comprising a collecting structure (4) for collecting the filtered fluid from the filter (2). An adhesive (5) holds the filter (2) in place with respect to the collecting structure (4). The filter (2), the substrate (3) with the collecting structure (4) and the adhesive (5) are arranged such that the collecting structure (4) contacts the filter (2) and the filter (2) is attached to the substrate (3) by the adhesive (5). The collecting structure is very close to the filter and the adhesive ensures that the collecting structure remains very close to the filter. The filtered fluid can therefore be collected very efficiently and very fast. The fluid filtering device is preferentially used for filtering blood in a biosensing device.

### FIELD OF THE INVENTION

The invention relates to a fluid filtering device, a sensing device for sensing a substance in a fluid, an analyzing device for analyzing a substance in a fluid, a fluid filtering method, and a manufacturing method for manufacturing a fluid filtering device.

### BACKGROUND OF THE INVENTION

KR 1020080051011 A discloses a micro filtration device for separation of blood plasma from the whole blood. The micro filtration device includes a whole blood inlet, a blood plasma outlet, a blood plasma storing chamber, a paper filter and a micro-structure. The blood plasma outlet is used to discharge the blood plasma separated from the flowing whole blood to the outside. The paper filter is formed between the whole blood inlet and the blood plasma storing chamber and separates the blood plasma from the whole blood. The micro-structure is formed inside the blood plasma storing chamber to move the blood plasma.

The micro filtration device has the drawback that without the help of external equipment like a syringe pump the filtering process takes a long time.

### SUMMARY OF THE INVENTION

It is an object of the present invention to provide a fluid filtering device and a fluid filtering method which allow filtering the fluid faster without the help of external equipment like a syringe pump.

In a first aspect of the present invention a fluid filtering device is presented, wherein the fluid filtering device comprises:
a filter for filtering a fluid,
a substrate comprising a collecting structure for collecting the filtered fluid from the filter,
an adhesive for holding the filter in place with respect to the collecting structure,

wherein the filter, the substrate with the collecting structure and the adhesive are arranged such that the collecting structure contacts the filter and the filter is attached to the substrate by the adhesive.

Since the collecting structure contacts the filter, wherein the filter is attached to the substrate by the adhesive for holding the filter in place, the collecting structure is very close to the filter and the adhesive ensures that the collecting structure remains very close to the filter. Moreover, since the collecting structure contacts the filter, i.e. since the adhesive is not arranged between the collecting structure and the filter at least at the contact site, the adhesive, which is generally hydrophobic, does generally not adversely influence the collection of the filtered fluid from the filter by the collecting structure. The filtered fluid can therefore be collected very efficiently and very fast.

The fluid is preferentially a bodily fluid, like blood, saliva, or urine. The filter is preferentially a blood filter, a saliva filter, or a urine filter. The filter has preferentially a porous polymer structure present in the form of a sheet of, for example, a few tenths of a mm thickness. The sheet is preferentially composed of micro-porous material. The filter surface is preferentially hydrophilic to achieve a good and fast wetting with the fluid.

The collecting structure is preferentially a micro structure being dimensioned such that the fluid is extracted out of the filter by capillary forces, if the fluid is a bodily fluid like blood, saliva or urine.

The contact between the collecting structure and the filter is preferentially a direct contact. The contact between the filter and the collecting structure can be at only a single contact site or at several contact sites.

The adhesive is preferentially a pressure-sensitive adhesive (PSA), which can be a double-sided tape, in particular, a double-sided pressure-sensitive adhesive tape.

The substrate is preferentially an injection-molded substrate.

It is preferred that at least a part of the collecting structure contacts the filter and that at least this part is hydrophilic.

It is further preferred that the fluid filtering device comprises a channel structure for guiding the fluid from the collecting structure to a sensing region for sensing the filtered fluid.

It is further preferred that the fluid filtering device comprises a sealing means for avoiding leakage of unfiltered fluid into the channel structure. Such sealing means can be a double-sided adhesive tape, which may surround the filter, a container wherein the filter forms the bottom of the container, et cetera.

The channel structure is preferentially a micro channel structure for driving filtered fluid by capillary forces.

It is also preferred that the substrate comprises an opening connecting the collecting structure with the channel structure for allowing the filtered fluid to flow from the collecting structure to the channel structure.

The opening, i.e. the transition from the collecting structure to the channel structure, is preferentially adapted such that the filtered fluid can be guided from the collecting structure to the channel structure by capillary forces, in particular, if the fluid is a bodily fluid like blood, saliva or urine.

It is further preferred that the fluid filtering device comprises a pin being located within the opening for forming a circumferential shape of the opening.

This further increases the capillary forces used for transferring the filtered fluid from the collecting structure to the channel structure.

It is further preferred that the channel structure is formed on a channel side of the substrate being opposite to a collecting side of the substrate on which the collecting structure is formed. Having the channel structure on the opposite side of the substrate allows the extraction of the filtered liquid to any spot outside the edge of the filter without having to traverse the filter edge in this way avoiding the risks of leakage from the filter into the channel structure and/or hampering of the capillary driven flow by geometrical transitions caused by the transition from the filter to a cover of the channel structure.

Preferentially a further substrate is arranged on the channel side for closing the channel structure.

It is further preferred that a contact region in which the collecting structure contacts the filter and an attachment region in which the filter is attached to the substrate by the adhesive are located in the same plane.

This ensures a very close arrangement of the filter and of the collecting structure, thereby further improving the extraction of filtered fluid out of the filter into the collecting structure.

The adhesive and the collecting structure have preferentially the same height with respect to the substrate for holding the filter in place and for having a contact between the collecting structure and the filter.

It is further preferred that the filter, the substrate with the collecting structure and the adhesive form a sandwich-like structure, wherein the adhesive is arranged between the filter and the substrate for holding the filter in place with respect to the collecting structure and wherein the adhesive and the collecting structure form an interleaved arrangement such that both the adhesive and the collecting structure contact the filter.

This allows to arrange the filter and the collecting structure very close to each other, wherein the filter is held in place by the adhesive and wherein the adhesive does not adversely affect the extraction of the filtered fluid out of the filter into the collecting structure. This allows also to have minimum dead volume between the filter and the channel structure and consequently fast delivery of filtered liquid to the channel structure.

In an embodiment, the interleaved arrangement of the adhesive and the collecting structure is formed by making islands of adhesive in between the collecting structure. The filter is then arranged on the islands of adhesive and on the collecting structure such that the filter is held in place by the islands of adhesive and the filter contacts the collecting structure. The collecting structure can comprise one or several inner openings and/or the collecting structure can be formed by separate protrusions formed on the substrate, wherein the islands of adhesive are arranged within the one or several inner openings of the collecting structure or between the separate protrusions of the collecting structure protruding from the substrate.

In a preferred embodiment, the adhesive comprises an inner opening, wherein the collecting structure protrudes through the inner opening for forming the interleaved arrangement.

It is further preferred that the inner opening of the adhesive surrounds the collecting structure, wherein the part of the collecting structure being surrounded by the inner opening of the adhesive and the inner opening of the adhesive have corresponding shapes.

This allows effectively holding the filter in place by the adhesive and extracting the filtered fluid out of the filter into the collecting structure.

The area of the filter covered by the adhesive does not extract filtered fluid, but the filtered fluid can flow inside the filter in a lateral direction to be extracted in the area of the filter being close to the collecting structure, in particular, contacting the collecting structure.

It is further preferred that the collecting structure comprises collecting channels, wherein the longitudinal open sides of the collecting channels face the filter for collecting the filtered fluid from the filter. The collecting channels are preferentially arranged radially.

In an embodiment, the collecting channels are arranged radially, wherein the filter, the substrate with the collecting structure and the adhesive form a sandwich-like structure, wherein the adhesive is arranged between the filter and the substrate for holding the filter in place with respect to the collecting structure, wherein the adhesive comprises an inner opening through which the collecting structure protrudes to contact the filter, wherein the shape of the inner opening of the adhesive corresponds to the radial arrangement of the collecting channels.

In a further embodiment, the collecting channels are arranged radially, wherein the filter, the substrate with the collecting structure and the adhesive form a sandwich-like structure, wherein the adhesive is arranged between the filter and the substrate for holding the filter in place with respect to the collecting structure, wherein the adhesive comprises an inner opening through which the collecting structure protrudes to contact the filter, wherein the collecting structure comprises a collecting surface protruding from a substrate surface of the substrate, wherein the collecting channels are formed as depressions in the collecting surface, wherein the inner opening of the adhesive corresponds to the circumference of the protruding collecting surface. In this embodiment, the collecting structure has preferentially a tart-like shape, wherein the protruding collecting surface with the depressions is shaped like being comprised of several substantially triangular pieces of a tart. The protruding collecting surface is preferentially circular and the adhesive has preferentially a corresponding round opening. This allows using an adhesive being less structured, thereby simplifying manufacturing the fluid filtering device.

It is further preferred that the channels are arranged to guide the collected filtered fluid to the opening of the substrate connecting the collecting structure with the channel structure.

The opening is preferentially located in the centre of a circle, wherein the collecting channels are adapted to guide the filtered fluid radially to the opening.

It is further preferred that the collecting channels are adapted such that at least one of the depth and the width of the collecting channels decreases towards the opening.

Preferentially, the collecting channels are adapted such that the smallest dimension, i.e. the depth or the width, of the respective collecting channel decreases towards the opening. This increases the capillary forces in the direction of the opening and therefore further improves the extraction of the filtered fluid out of the filter into the collecting structure and also improves the guidance of the filtered fluid from the collecting structure to the channel structure. It also reduces the probability of enclosing air bubbles inside the filtered liquid which would hamper the flow inside the channel structure.

The collecting structure and the channel structure can be an integrated structure, wherein the collecting structure can be defined as the part of the integrated structure being close to the filter and the further part of the integrated structure can be regarded as the channel structure for guiding the filtered fluid from the collecting structure to, for example, a sensing region for sensing the fluid, in particular, for sensing a substance within the fluid.

It is further preferred that at least a part of the substrate with the collecting structure is surface treated for reducing the contact angle between the filtered fluid and the surface of the part of the substrate with the collecting structure.

The surface treatment can, for example, be performed by using a plasma like an oxygen plasma. A reduced contact angle between the filtered fluid and the surface of the part of the substrate with the collecting structure increases the capillary forces, thereby further improving the extraction of the filtered fluid out of the filter into the collecting structure.

It is further preferred that the fluid filtering device comprises a venting structure for venting the collecting structure.

The venting structure allows a gas like air leaving the collecting structure in order to facilitate the introduction of the filtered fluid into the collecting structure.

The venting structure preferentially comprises a venting channel at an end of the collecting channels, wherein the collecting channels comprise a sharp edge at this end of the collecting channels such that bodily fluids are not capillary forcable into the venting channel. The cross-section of the venting channel is preferentially larger than the cross-section of the channel structure leading to preferentially the sensing part.

This reduces the probability that the filtered fluid leaves the collecting channels through the venting channel.

It is further preferred that the venting structure comprises a venting channel at an end of the collecting structure, wherein the collecting structure comprise a hydrophobic end region at this end of the collecting structure. In particular, the venting channel is located at an end of collecting channels of the collecting structure, wherein the collecting channels comprise a hydrophobic end region at this end of the collecting channels.

Also this reduces the probability that the filtered fluid leaves the collecting channels through the venting channel.

The venting channel is preferentially a venting channel enclosing the collecting structure and being connected to the ends of the collecting structure being opposite to the ends of the collecting structure at the opening to the channel structure. In particular, the venting channel encloses collecting channels of the collecting structure, wherein the venting channel is connected to the ends of the collecting channels being opposite to the ends of the collecting channel at the opening to the channel structure. From the venting channel preferentially a further channel guides the vented gas to the outside of the filtering device.

In a further aspect of the present invention a sensing device for sensing a substance in a fluid is presented, wherein the sensing device comprises:
a fluid filtering device as defined in claim 1 for filtering the fluid,
a sensing region being adapted for sensing the substance in the filtered fluid,
a channel structure for guiding the filtered fluid from the filtering device to the sensing region.

The sensing device is preferentially a disposable cartridge, wherein for analyzing the fluid in the sensing region the sensing device is introduced into an analyzing device for analyzing a substance in the filtered fluid in the sensing region. Preferentially, after the sensing device has been used, it can be discarded, and the analyzing device is not discarded, i.e. the analyzing device is used several times for analyzing filtered fluid in sensing regions of different sensing devices. In another embodiment, also the sensing device itself can comprise an analyzing unit for analyzing the fluid, in particular, a substance within the fluid, in the sensing region.

In a further aspect of the present invention an analyzing device for analyzing a substance in a fluid is presented, wherein the analyzing device is adapted to cooperate with a sensing device as defined in claim 12 for analyzing the substance in the fluid, the analyzing device comprises:
a sensing device receiving section for receiving the sensing device,
an analyzing unit for analyzing the substance in the filtered fluid in the sensing region of the sensing device, if the sensing device is received in the sensing device receiving section.

The analyzing unit can be adapted to perform a sandwich assay or another kind of assay. For example, magnetic beads can be attached to the substance to be analyzed in the filtered fluid, wherein the magnetic beads with the attached substance can be attracted to a sensing surface of the sensing region by magnetic forces. The analyzing unit can be adapted to analyze the concentration of magnetic beads on the sensing surface of the sensing region, in order to determine the concentration of the substance in the filtered fluid. The analyzing result is then, for example, the concentration of the substance in the filtered fluid.

In a further aspect of the present invention a fluid filtering method is presented, wherein a fluid is filtered by a filter and the filtered fluid is collected by a collecting structure, wherein the filter is held in place with respect to the collecting structure by an adhesive and wherein the filter, the substrate with the collecting structure and the adhesive are arranged such that the collecting structure contacts the filter and the filter is attached to the substrate by the adhesive.

In a further aspect of the present invention a manufacturing method for manufacturing a fluid filtering device is presented, wherein the manufacturing method comprises following steps:
providing a filter for filtering a fluid, a substrate comprising a collecting structure for collecting the filtered fluid from the filter, and an adhesive for holding the filter in place with respect to the collecting structure,
arranging the filter, the substrate with the collecting structure and the adhesive such that the collecting structure contacts the filter and the filter is attached to the substrate by the adhesive.

It shall be understood that the fluid filtering device of claim 1, the sensing device of claim 12, the analyzing device of claim 13, the fluid filtering method of claim 14 and the manufacturing method of claim 15 have similar and/or identical preferred embodiments as defined in the dependent claims.

It shall be understood that a preferred embodiment of the invention can also be any combination of the dependent claims with respect to the independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily a fluid filtering device,

FIG. 2 shows schematically and exemplarily a sensing device,

FIG. 3 shows schematically and exemplarily an analyzing device,

FIG. 4 shows schematically and exemplarily a cross section through a fluid filtering device,

FIG. 5 shows schematically and exemplarily a part of a fluid filtering device,

FIG. 6 shows schematically and exemplarily a collecting structure and a venting structure,

FIGS. 7 and 8 show schematically and exemplarily an opening connecting a collecting structure with a channel structure,

FIG. 9 shows schematically and exemplarily a part of a further fluid filtering device,

FIG. 11 shows a flowchart exemplarily illustrating a fluid filtering method, and

FIG. 10 shows schematically and exemplarily a collecting structure and a venting structure,

FIG. 12 shows a flowchart exemplarily illustrating a manufacturing method for manufacturing a fluid filtering device.

### DETAILED DESCRIPTION OF EMBODIMENTS

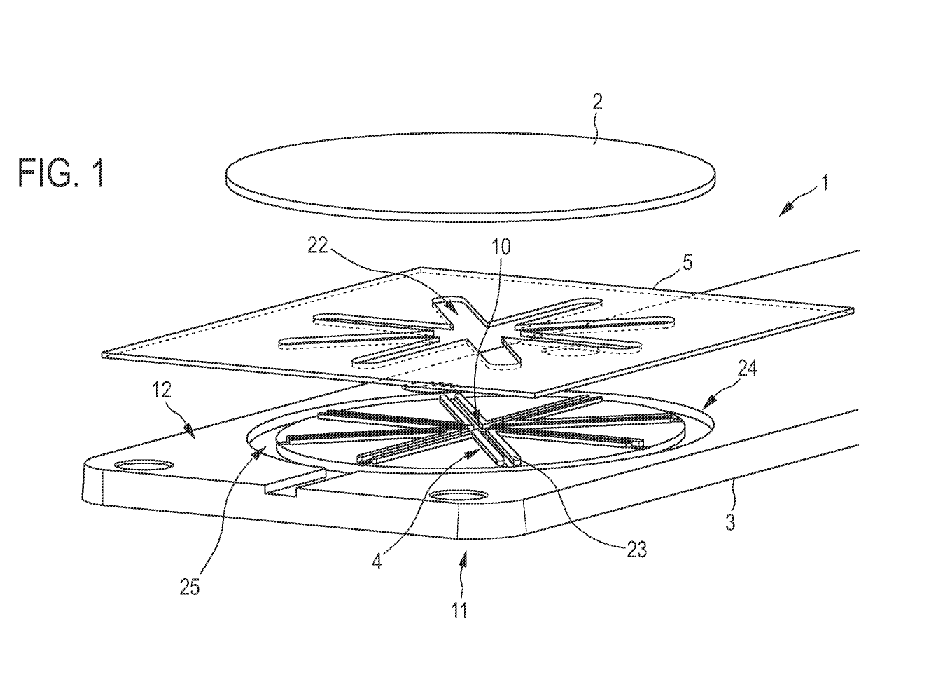

FIG. 1 shows schematically and exemplarily a fluid filtering device 1. The fluid filtering device 1 comprises a filter 2 for filtering a fluid and a substrate 3 comprising a collecting structure 4 for collecting the filtered fluid from the filter 2. The fluid filtering device 1 further comprises an adhesive 5 for holding the filter 2 in place with respect to the collecting structure 4. The filter 2, the substrate 3 with the collecting structure 4 and the adhesive 5 are arranged such that the collecting structure 4 is close to the filter 2 and the filter 2 is attached to the substrate 3 by the adhesive 5. The collecting structure 4 contacts the filter 2 at at least one contact side.

The filter 2 is a blood filter comprising porous polymer structures present in the form of a sheet of a few tenths of a mm thickness. The fluid, which is preferentially blood, has to be placed on the surface of the filter 2 being in FIG. 1 the top surface of the filter 2. This top surface is hydrophilic to achieve a good and fast wetting with the blood sample.

The substrate 3 with the collecting structure 4 is an injection-molded plastic substrate. In other embodiments, the substrate can be made of another material. Moreover, the collecting structure and the substrate can be made of the same material or they can be made of different materials. In this embodiment, the adhesive 5 is a double-sided pressure-sensitive adhesive tape. In another embodiment, another kind of adhesive can be used for holding the filter 2 in place with respect to the collecting structure 4 and for attaching the filter 2 to the substrate 3.

At least the parts of the collecting structure 4 being close to the filter 2 are hydrophilic. Preferentially, the entire collecting structure 4 is hydrophilic.

The collecting structure 4 is a micro structure being dimensioned such that the fluid is extracted out of the filter 2 by capillary forces, if the fluid is a bodily fluid like blood, saliva or urine. The height of the collecting structure is preferably between 10 and 1000 micrometers, more preferably between 20 and 300 micrometers

The collecting structure 4 contacts the filter 2 directly.

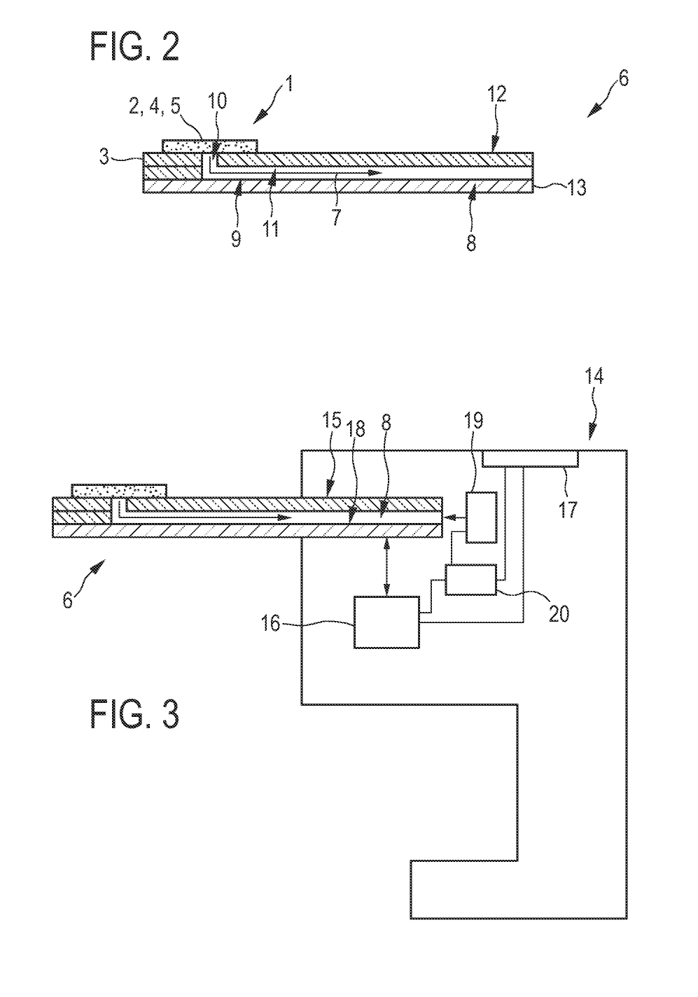

The fluid filtering device further comprises a channel structure 9 for guiding the fluid from the collecting structure 4 to a sensing region 8 for sensing the filtered fluid, wherein the channel structure 9 is formed on a channel side 11 being opposite to a collecting side 12 of the substrate 3 on which the collecting structure 4 is formed. The channel structure 9 is schematically and exemplarily shown in FIG. 2.

FIG. 2 shows schematically and exemplarily an embodiment of a sensing device 6 for sensing a substance in a fluid. The sensing device 6 comprises the fluid filtering device 1 with the channel structure 9 and a sensing region 8 being adapted for sensing the substance in the filtered fluid. The channel structure 9 can also be regarded as not being a part of the fluid filtering device, but as a part of the sensing device. The collecting structure 4 and the channel structure 9 are connected to each other such that filtered fluid can flow from the collecting structure 4 to the channel structure 9 in the direction indicated by the arrow 7 in FIG. 2.

For clarity reason, in FIG. 2 the details of the fluid filtering device 1 shown in FIG. 1, in particular, the filter 2, the collecting structure 4, and the adhesive 5 are indicated by a single box only. It should be noted that the sensing device described above with reference to FIG. 2 can have further elements which are not described above. For example, the sensing device 6 can comprise a venting means like a venting opening behind the sensing region 8 with respect to the flowing direction indicated by the arrow 7. This can further increase the velocity of transferring the fluid to the sensing region 8. The venting means is, for example, a venting hole in the substrate 3.

A further substrate 13 is arranged on the channel side 11 of the substrate 3 for closing the channel structure 9.

The sensing device 6 is a disposable cartridge which is introduceable in a sensing device receiving section of an analyzing device for analyzing a substance in the filtered fluid present in the sensing region 8.

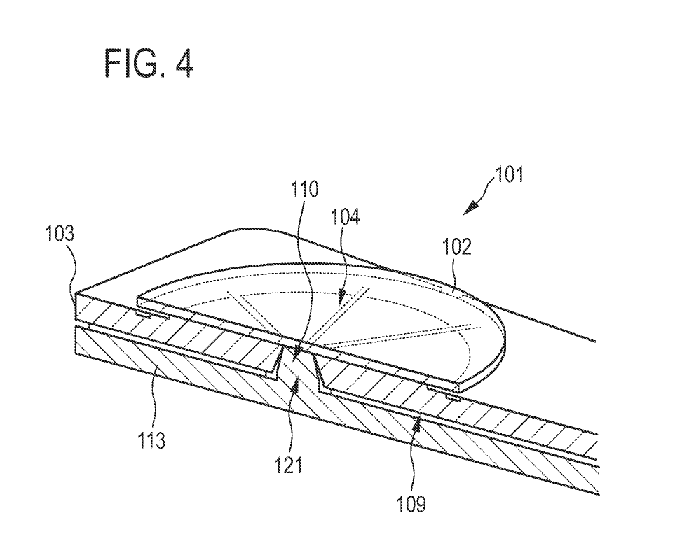

An embodiment of an analyzing device 14 is schematically and exemplarily shown in FIG. 3.

The analyzing device 14 for analyzing a substance in a fluid is adapted to cooperate with the sensing device 6 for analyzing the substance in the fluid. The analyzing device 14 comprises a sensing device receiving section 15 for receiving the sensing device such that an analyzing unit 16 of the analyzing device 14 can analyze the substance in the filtered fluid in the sensing region 8 of the sensing device 6, if the sensing device 6 is introduced into the sensing device receiving section 15. The analyzing device 14 further comprises a display unit 17 for displaying the result of the analysis to a user.

In this embodiment, the sensing region 8 and the analyzing unit 17 are adapted to perform a sandwich assay. Magnetic beads which can be attached to the substance to be analyzed are provided in the sensing region 8 of the sensing device 6. The magnetic beads attach to the substance and the magnetic beads with the attached substance are attracted to a sensing surface 18 of the sensing region 8 by magnetic forces. The magnetic forces are preferentially generated by a magnetic forces generating unit 19 which is also schematically shown in FIG. 3 and which is, for example, an electro magnet.

The analyzing device 14 further comprises a control unit 20 for controlling the analyzing unit 16, the magnetic forces generating unit 19 and the display 17.

The sensing surface 18 is adapted such that magnetic beads with the substance are bound to the sensing surface 18, after they have been attracted to the sensing surface 18. The analyzing unit 16 is adapted to determine the concentration of magnetic beads on the sensing surface 18 of the sensing region 8, in order to determine the concentration of the substance in the filtered fluid. The analyzing result is, in this embodiment, the concentration of the substance in the filtered fluid, which is presented to a user via the display unit 17. The concentration of the magnetic beads on the sensing surface 18 can be determined by known determination methods which, for example, measure the concentration of magnetic beads on the sensing surface 18 magnetically or optically, in particular, by using frustrated total internal reflection.

In other embodiments, the analyzing unit 16 and the sensing region 8 can be adapted to perform another kind of assay for determining a property of the fluid like the concentration of a substance within the fluid.

The channel structure 9 is a micro channel structure for driving the filtered fluid, which is preferentially blood plasma, by capillary forces from the collecting structure 4 to the sensing region 8.

Referring again to FIG. 2, the substrate 3 comprises an opening 10 connecting the collecting structure 4 with the channel structure 9 for allowing the filtered fluid to flow from the collecting structure 4 to the channel structure 9.

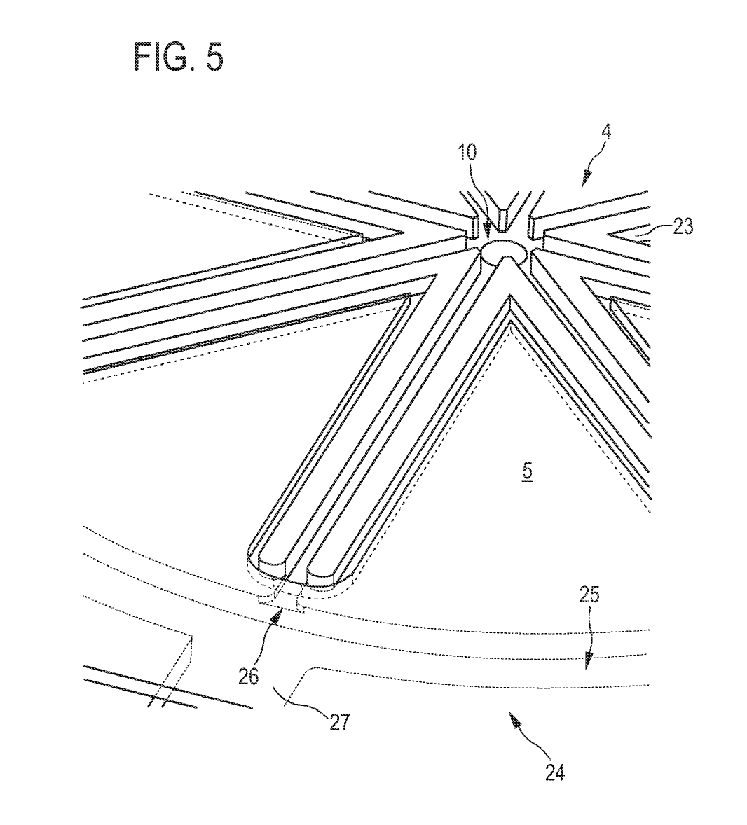

The opening 10, i.e. the transition from the collecting structure 4 to the channel structure 9, is adapted such that the filtered fluid can be guided from the collecting structure 4 to the channel structure 9 by capillary forces, if the fluid is a bodily fluid like blood, saliva or urine. For increasing the capillary forces, in an embodiment, the fluid filtering device comprises a pin being located within the opening for forming a circumferential shape of the opening. This is exemplarily and schematically shown in FIG. 4.

FIG. 4 shows schematically and exemplarily a cross section through an embodiment of a fluid filtering device 101. The fluid filtering device 101 comprises a filter 102 arranged on a collecting structure 104 provided on a substrate 103. The fluid filtering device 101 comprises a channel structure 109 for guiding the fluid from the collecting structure 104 to a sensing region for sensing the filtered fluid. The substrate 103 comprises an opening 110 connecting the collecting structure 104 with the channel structure 109 for allowing the filtered fluid to flow from the collecting structure 104 to the channel structure 109, wherein a pin 121 is located within the opening 110 for forming a circumferential shape of the opening 110. The pin has preferentially the shape of a cone and the opening 110 has preferentially a corresponding inverse shape. The pin 121 is a part of a further substrate 113 which forms the channel structure 109 together with the substrate 103.

Referring again to FIG. 1, it can be seen that, if the different elements shown in FIG. 1 in an exploded view are put together, a contact region in which the collecting structure 4 contacts the filter 2 and an attachment region in which the filter 2 is attached to the substrate 3 by the adhesive 5 are located in the same plane. This ensures a very close arrangement of the filter 2 and of the collecting structure 4, thereby improving the extraction of filtered fluid out of the filter 2 into the collecting structure 4.

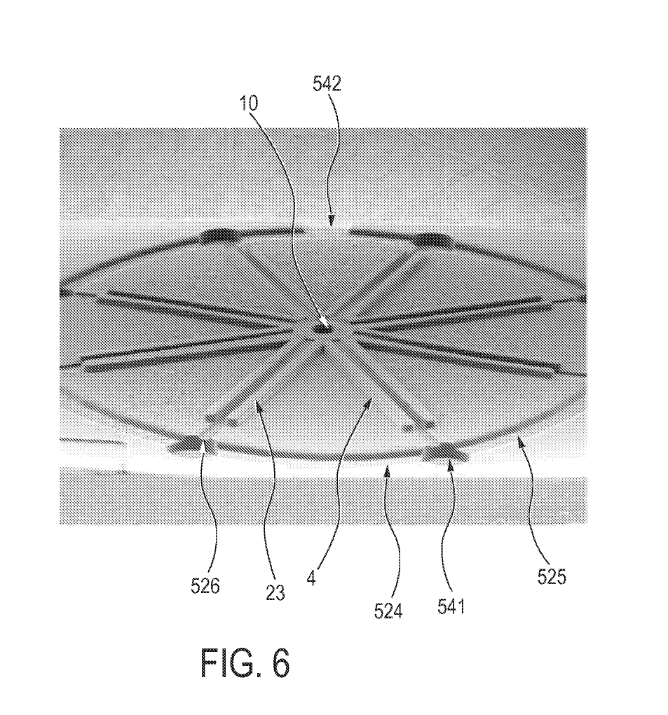

The filter 2, the substrate 3 with the collecting structure 4 and the adhesive 5 form a sandwich-like structure, wherein the adhesive 5 is arranged between the filter 2 and the substrate 3 for holding the filter 2 in place with respect to the collecting structure 4 and wherein the adhesive 5 comprises an inner opening 22 through which the collecting structure 4 protrudes to contact the filter 2 for forming an interleaved arrangement 40 (see FIG. 5). This arranges the filter 2 and the collecting structure 4 very close to each other, wherein the filter 2 is held in place by the adhesive 5 and wherein the adhesive 5 does not adversely affect the attraction of the filtered fluid out of the filter 2 into the collecting structure 4.

The inner opening 22 of the adhesive 5 surrounds the collecting structure 4 and the part of the collecting structure 4 being surrounded by the inner opening 22 of the adhesive 5 and the inner opening 22 of the adhesive 5 have corresponding shapes.

The collecting structure 4 comprises collecting channels 23, wherein the longitudinal open sides of the collecting channels 23 face the filter 2, in particular, at least at a contact site a longitudinal open side of a collecting channel 23 contacts the filter 2, for collecting the filtered fluid from the filter 2. The collecting channels 23 are arranged radially and guide the filtered fluid to the opening 10. At least one of the depth or the width of the collecting channels 23 decreases towards the opening 10. In this embodiment, the depth of the collecting channels 23 decreases towards the opening 10. In another embodiment, also the width or only the width of the collecting channels 23 can decrease in the direction towards the opening 10. In an embodiment, parts of the fluid filtering device, which can come into contact with the fluid in operation, for example the collecting structure 4 and the channel structure 9, are surface treated for reducing the contact angle between the fluid and the surface of these parts. The surface treatment is preferentially performed by using oxygen plasma.

The fluid filtering device 1 further comprises a venting structure 24 for venting the collecting structure 4. The venting structure 24 allows a gas like air leaving the collecting structure 4 in order to facilitate the introduction of the filtered fluid into the collecting structure 4. The venting structure 24 comprises a venting channel 25 arranged at the ends of the collecting channels 23 opposite to the opening 10. The venting structure 24 comprises a sharp edge 26 at the respective end of the collecting channels 23 such that bodily fluids like blood, saliva or urine are not capillary forceable into the venting channel 25. The sharp edge 26 is schematically and exemplarily shown in FIG. 5. The sharp transition 26 is provided to act as a fluidic stop. In this way only gas like air but no fluid like blood plasma can escape from the collecting channels 23 to the venting channel 25. In addition or alternatively, the respective ends of the collecting channels 23 being opposite to the opening 10 can be hydrophobic for reducing the probability that the filtered fluid leaves the collecting channels 23 through the venting channel 25

The venting channel 25 is a circular venting channel enclosing the collecting channels 23, wherein the circular venting channel 25 is connected to the ends of the collecting channels 23 being opposite to the ends of the collecting channels 23 at the opening 10 to the channel structure 9. A further venting channel 27 guides the vented gas from the circular venting channel 25 to the outside of the filtering device 1.

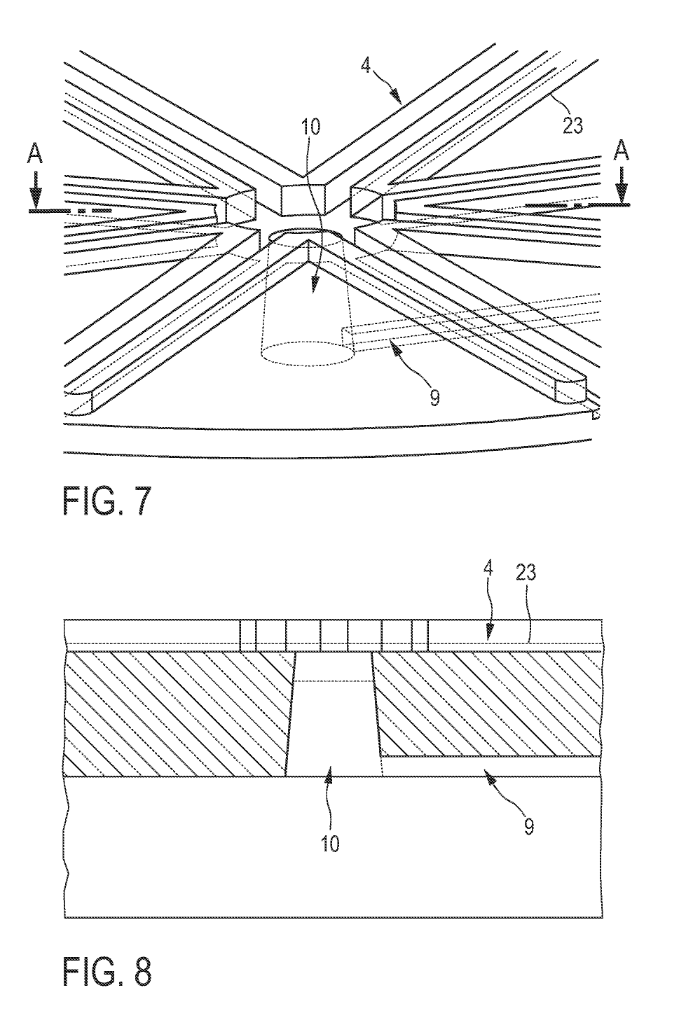

FIG. 6 shows schematically and exemplarily the collecting structure 4 with the collecting channels 23 and with a further embodiment of a venting structure 524. Also the venting structure 524 comprises a venting channel 525 arranged at the ends of the collecting channels 23 opposite to the opening 10. The venting structure 524 comprises a sharp edge 526 at the respective end of the collecting channels 23 such that body fluids like blood, saliva or urine are not capillary forceable into the venting channel 525. In addition or alternatively, the respective ends of the collecting channels 23 being opposite to the opening 10 can be hydrophobic for reducing the probability that the filtered fluid leaves the collecting channels 23 through the venting channel 525. Moreover, the venting structure 524 shown in FIG. 6 comprises a venting opening 542 which allows the vented gas to leave the circular venting channel 525.

The venting structure 524 comprises depressions within the venting channel 525 at the ends of the collecting channels 23 being opposite to the ends at the opening 10. The depressions 526 can be circular and have a diameter being larger than the width of the venting channel 525.

The collecting structure 4 with the collecting channels 23 and the venting structure 524 are preferentially used together with the further components like the filter and the adhesive described above, in particular, with reference to FIGS. 1 and 5.

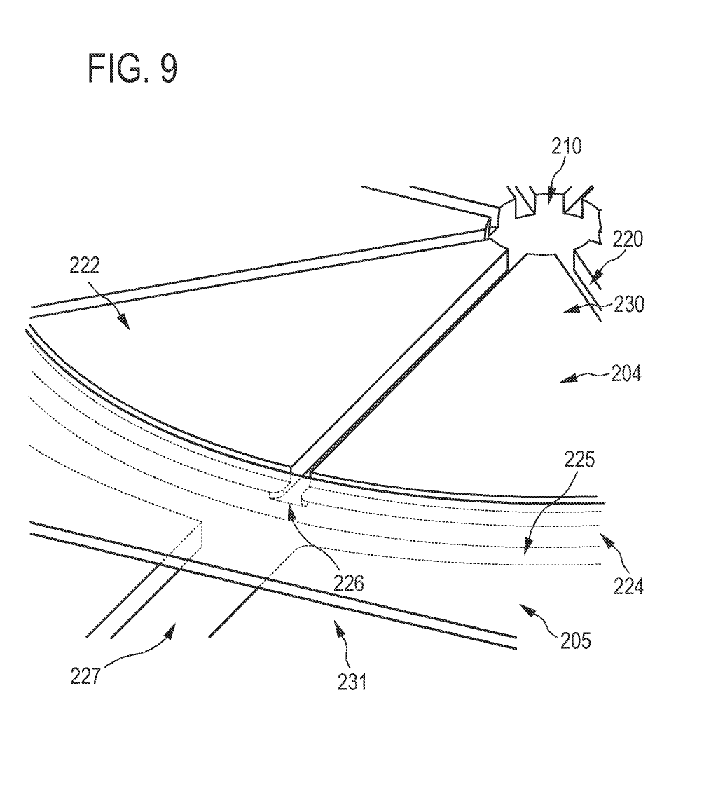

FIG. 7 shows schematically and exemplarily the connection of the collecting channels 23 of the collecting structure 4 to the channel structure 9 via the opening 10 in more detail. FIG. 8 shows schematically and exemplarily a cross section along the line A-A in FIG. 7.

In FIGS. 7 and 8 not all elements of the fluid filtering device are shown, but only the elements needed for illustrating the connection between the collecting channels 23 of the collecting structure 4 and the channel structure 9 via the opening 10.

The collecting channels 23 of the collecting structure 4 together with the circular venting channel 25 form a wheel-like structure in the substrate 3 which is aligned with the structured adhesive tape 5 such that the elevated collecting channels 23 protrude through the inner opening 22 in the adhesive tape 5 to contact the filter 2. The filter 2 is held in place by the adhesive tape 5. The area of the filter 2 covered by the adhesive tape 5 does not extract fluid like blood plasma, but the fluid can flow inside the filter 2 in the lateral direction to be extracted in the area of the filter 2 close to the collecting channels 23.

The distance between the “spokes” of the wheel-like structure as well as the number of spokes and the width of the spokes can be adjusted to the type of filter which is used to optimize filter efficiency and speed. As already mentioned above, the molded structures in the substrate 3 are preferentially surface treated for a low contact angle to the extracted fluid like blood plasma to have high capillary force and consequently a high extraction speed.

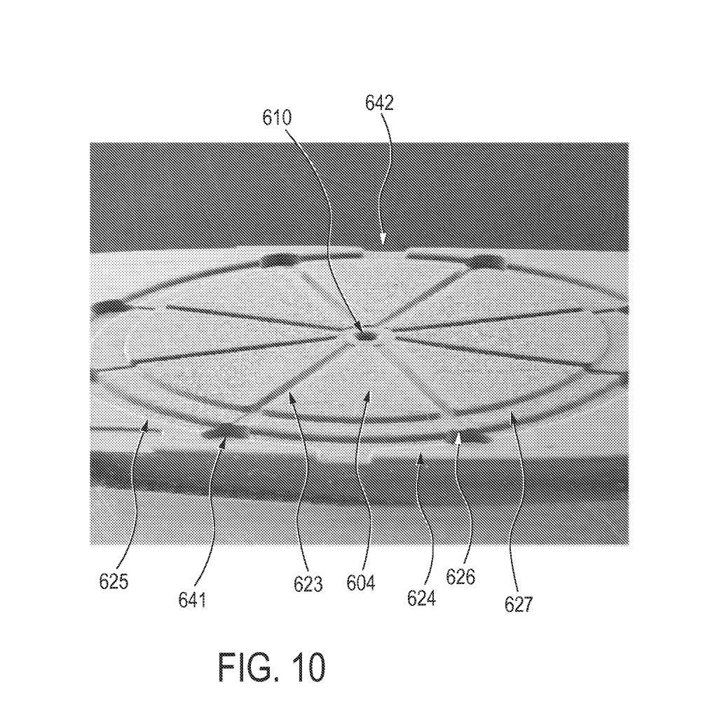

In a further embodiment of the fluid filtering device the structure of the adhesive is much less detailed. The collecting structure, the adhesive and the venting structure of this embodiment are partially schematically and exemplarily shown in FIG. 9.

Also the collecting structure 204 shown in FIG. 9 comprises collecting channels 223, wherein the longitudinal open sides of the collecting channels 223 face the filter (not shown in FIG. 9) for collecting the filtered fluid from the filter. The collecting channels 223 are arranged radially, wherein the filter, the substrate with the collecting structure 204 and the adhesive 205 form a sandwich-like structure. The adhesive 205 is arranged between the filter and the substrate for holding the filter in place with respect to the collecting structure 204, wherein the adhesive 205 comprises an inner opening 222 through which the collecting structure 204 protrudes to contact the filter and to form an interleaved arrangement 240. The collecting structure 204 comprises a collecting surface 230 protruding from a substrate surface 231 facing the filter. The collecting channels 223 are formed as depressions in the collecting surface 230, wherein the inner opening 222 of the adhesive 205 corresponds to the circumference of the protruding collecting surface 230.

The collecting structure 204 has a tart-like shape, wherein the protruding collecting surface 230 with the depressions 223 is shaped like being comprised of several substantially triangular pieces of a tart. The protruding collecting surface 230 is circular and the adhesive 205, which is preferentially a double-sided pressure-sensitive adhesive tape, has a corresponding round inner opening 222.

If the filter is arranged on the adhesive 205 shown in FIG. 9, the filter is only attached to the adhesive 205 at the circumference. This allows a faster extraction, wherein the contact to the underlying collecting structure 204 is still well controlled.

The collecting channels 223 are vented by a venting structure 224. The venting structure 224 shown in FIG. 9 corresponds to the venting structure 24 shown in FIGS. 1 and 5. Gas like air can leave the collecting channels 223 at their ends being opposite to their ends at the opening 210. The gas leaves the collecting channels 223 into a circular venting channel 225 surrounding the tart-like collecting structure 204. A further venting channel 227 connects the circular venting channel 225 with the outside of the fluid filtering device. Also in this embodiment the venting structure comprises a sharp edge 226 at the end of the collecting channels 223 facing the circular venting channel 225 for providing a fluidic stop such that a gas can leave the collecting channels 223 but not the filtered fluid.

FIG. 10 shows schematically and exemplarily an embodiment of a collecting structure 604 and of a venting structure 624 which can be used in the fluid filtering device. The collecting structure 604 comprises collecting channels 623 which are radially arranged towards an opening 610 to a channel structure. The collecting channels 623 are vented by the venting structure 624. Gas like air can leave the collecting channel 623 at their ends being opposite to their ends at the opening 610. The gas leaves the collecting channels 623 into a circular venting channel 625 surrounding the tart-like collecting structure 604. The gas can leave the venting channel 625 via venting openings 642. Also in this embodiment the venting structure comprises a sharp edge 626 at the end of the collecting channels 623 facing the circular venting channel 625 for providing a fluidic stop such that gas can leave the collecting channel 623 but not the filtered fluid.

Between the collecting structure 604 and the venting channel 625 an area 627 of the substrate is present, where the adhesive is to be placed. Also in this embodiment the adhesive, which is preferentially an adhesive tape, bridges the venting channel, if the adhesive is placed on the area 627 of the substrate. The collecting channels 623 extend from the collecting structure underneath the adhesive into the venting channel 625. Moreover, also in this embodiment the venting structure 624 comprises depressions 641 at the ends of the collecting channels 623 being opposite to the ends of the collecting channels 623 at the opening 610, wherein the depressions 641 have a larger depth than the venting channel 625. Moreover, the depressions 641 can be circular and have a larger diameter than the width of the collecting channel 623 and preferably also larger than the venting channel 625. This venting structure 624 further reduces the probability that filtered fluid leaves the collecting structure 604 into the venting channel 625 and not into the opening 610. The collecting structure 604 and the venting structure 624 are preferentially used together with the adhesive and the further elements of the fluid filtering device described above, in particular, with respect to FIG. 9.

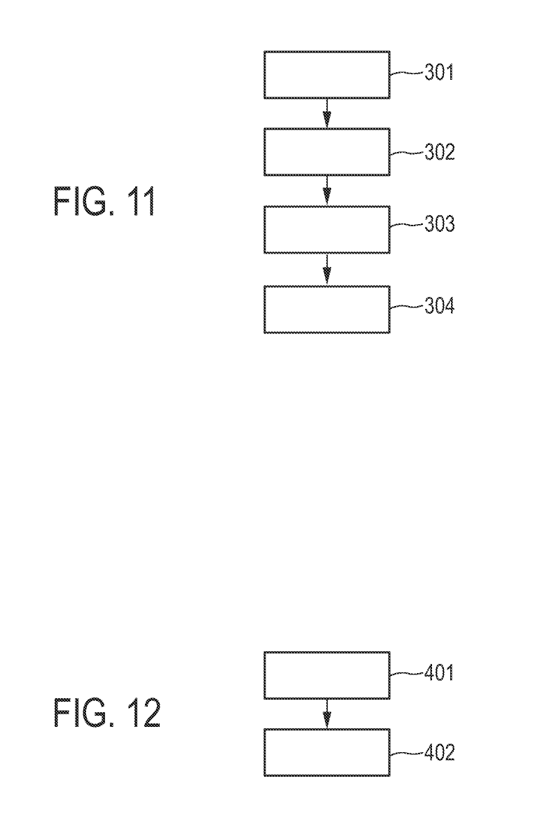

In the following an embodiment of a fluid filtering method will be exemplarily described with reference to a flowchart shown in FIG. 11.

In step 301, a fluid sample is provided on the filter of the fluid filtering device. In step 302, the fluid sample is filtered by the filter, and in step 303 the filtered fluid is collected by the collecting structure, wherein the filter is held in space with respect to the collecting structure by the adhesive and wherein the filter, the substrate with the collecting structure and the adhesive are arranged such that the collecting structure contacts to the filter and the filter is attached to the substrate by the adhesive.

In step 304, the filtered fluid is transferred from the collecting structure to the channel structure via the opening by capillary forces and further to the sensing region.

In the following an embodiment of a manufacturing method for manufacturing a fluid filtering device will exemplarily be described with reference to a flowchart shown in FIG. 12.

In step 401, a filter for filtering a fluid, a substrate comprising a collecting structure for collecting the filtered fluid from the filter, and an adhesive for holding the filter in place with respect to the collecting structure are provided. In step 402, the filter, the substrate with the collecting structure and the adhesive are arranged such that the collecting structure contacts the filter and the filter is attached to the substrate by the adhesive.

Different transitions between the collecting channels and the opening to the channel structure can be present. For example, a distance can be present between the end of the collecting channels facing the opening and the opening itself as shown, for example, in FIG. 5. However, these ends can also extend to the edge of the opening. Moreover, the opening can be surrounded by an elevated area having a height being smaller than the height of the top ends of the collecting channels as shown in FIGS. 6 and 10. These different transitions between the collecting channels and the opening to the channel structure can be combined with any of the above described fluid filtering devices, in particular, with any of the above described filters, adhesives and venting structures.

The fluid filtering device can allow for an efficient plasma extraction from blood filters integrated in a cartridge for a single use, which can be manufactured easily and at low cost. An easy and reliable connection of the filter can be achieved while still using the available filter capacity in the best way.

The sensing device alone or together with the analyzing device is preferentially a biosensor for the detection of specific components in body fluids like saliva, urine, blood. The biosensor makes preferentially use of magnetic beads covered with antibodies and of specific magnetic actuation protocols to optimize the assay performance. The presence of a substance like target molecules in the fluid is preferentially detected by a binding or prohibited binding of magnetic beads to the sensing surface of the sensing region of the sensing device covered with specific antibodies. The presence of the magnetic beads bound to the sensing surface can be detected by the analyzing unit by using optical and/or magnetic means. If a substance in blood has to be detected, the fluid filtering device removes cells present in the blood, in order to prevent that the magnetic beads get adsorbed to the cells.

The fluid filtering device is preferentially adapted to work with a minute amount of fluid sample. The fluid filtering device integrates a filter like a blood filter into a sensing device by making use of a combination of an adhesive, in particular, an adhesive tape, and a micro structure, which is preferentially made of plastic and which comprises the collecting structure and preferentially also the channel structure, such that a good physical contact of the filter is achieved without mechanical support and a fast and efficient fluid extraction, in particular, blood plasma extraction, is achieved by hydrophilic collecting structures which contact with the filter and conduct the filtered fluid in the most efficient way by capillary forces to the channel structure. The fluid filtering device is preferentially designed such that the efficiency and the capacity of the filter are not compromised by making use of the lateral permeability of the filter. The adhesive is preferentially structured and aligned with the fluidic structures like the collecting structure on the substrate.

Although certain embodiments have been described, other embodiments, in particular, having other shapes of adhesive and collecting structure, can be designed with a similar performance. In an embodiment, the inner opening in the adhesive and the molded substrate, in particular, the collecting structure, are aligned, wherein the height of structures on the molded substrate is substantially equal to the thickness of the adhesive and wherein the structures on the substrate, i.e. the collecting structures, provide capillary force to pull the fluid out of the filter, which is located on top of the adhesive, and into one or more openings present in the substrate. More than one substrate can be stacked underneath the adhesive, in particular, underneath the adhesive tape, to achieve the desired fluid routing for the cartridge.

Although certain embodiments have been described separately, features of the embodiments can be combined to a further embodiment. For example, each of the above described embodiments can comprise a pin in the opening connecting the collecting structure and the channel structure.

Although in the above described embodiments the collecting structure and the channel structure are formed on opposite sides of a substrate, the collecting structure and the channel structure can also be formed on the same side of the substrate.

It should be noted that not all figures show all elements of the filtering device, the sensing device or the analyzing device, respectively, for clarity reason. For example, although FIGS. 5 and 8 do not show the filter, also the filter is of course present in the fluid filtering device.

The sensing device can be any suitable sensing device to detect the presence of magnetic particles, on or near to the sensing surface, based on any property of the particles, for example it can detect via magnetic methods (for example, magnetoresistive, Hall, coils), optical methods (for example imaging, fluorescence, chemiluminescence, absorption, scattering, evanescent field techniques, surface plasmon resonance, Raman, et cetera), sonic detection (surface acoustic wave, bulk acoustic wave, cantilever, quartz crystal et cetera), electrical detection (for example conduction, impedance, amperometric, redox cycling), combinations thereof, et cetera.

The sensing device can be a sensing device for the detection of molecular targets. Molecular targets often determine the concentration and/or presence of larger moieties, for example, cells, viruses, or fractions of cells or viruses, tissue extract, et cetera.

The analyzing unit can provide measurement data as an end-point measurement, as well as by recording signals kinetically or intermittently. The particles like the magnetic beads can be further processed prior to detection. An example of further processing is that materials are added or that the (bio)chemical or physical properties of the label, i.e. of the magnetic beads, are modified to facilitate detection.

The devices and methods described above can be used with several biochemical assay types, for example binding/unbinding assay, sandwich assay, competition assay, displacement assay, enzymatic assay, et cetera. The devices and methods described above are suited for sensor multiplexing, i.e. the parallel use of different sensors and sensor surfaces, label multiplexing, i.e. the parallel use of different types of labels, and chamber multiplexing, i.e. the parallel use of different reaction chambers. The sensing device described above can be used as rapid, robust, and easy to use point-of-care biosensors for small sample volumes. They can preferentially be used in automated high-throughput testing.

The magnetic beads are preferentially nano-particles having at least one dimension ranging between 3 nm and 5000 nm, preferably between 10 nm and 3000 nm, more preferred between 50 nm and 1000 nm.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Any reference signs in the claims should not be construed as limiting the scope.

The present invention is related to a fluid filtering device comprising

a filter for filtering a fluid and a substrate comprising a collecting structure for collecting the filtered fluid from the filter. An adhesive holds the filter in place with respect to the collecting structure. The filter, the substrate with the collecting structure and the adhesive are arranged such that the collecting structure contacts the filter and the filter is attached to the substrate by the adhesive The collecting structure is very close to the filter and the adhesive ensures that the collecting structure remains very close to the filter. The filtered fluid can therefore be collected very efficiently and very fast. The fluid filtering device is preferentially used for filtering blood in a biosensing device.

1. A fluid filtering device comprising:
a filter (2) for filtering a fluid,
a substrate (3) comprising a collecting structure (4) for collecting the filtered fluid from the filter (2),
an adhesive (5) for holding the filter (2) in place with respect to the collecting structure (4),
wherein the filter (2), the substrate (3) with the collecting structure (4) and the adhesive (5) are arranged such that the collecting structure (4) contacts the filter (2) and the filter (2) is attached to the substrate (3) by the adhesive (5).

2. The fluid filtering device as defined in claim 1, wherein the fluid filtering device (1) further comprises a channel structure (9) for guiding the fluid from the collecting structure (4) to a sensing region (8) for sensing the filtered fluid, wherein the channel structure (9) is formed on a channel side (12) of the substrate (3) being opposite to a collecting side (11) of the substrate (3) on which the collecting structure (4) is formed.

3. The fluid filtering device as defined in claim 1, wherein a contact region in which the collecting structure (4) contacts the filter (2) and an attachment region in which the filter is attached to the substrate (3) by the adhesive (5) are located in the same plane.

4. The fluid filtering device as defined in claim 1, wherein the filter (2), the substrate (3) with the collecting structure (4) and the adhesive (5) form a sandwich-like structure, wherein the adhesive (5) is arranged between the filter (2) and the substrate (3) for holding the filter (2) in place with respect to the collecting structure (4) and wherein the adhesive (5) and the collecting structure (4) form an interleaved arrangement (40; 240) such that both the adhesive (5) and the collecting structure (4) contact the filter (2),

5. The fluid filtering device as defined in claim 4, wherein the adhesive (5) comprises an inner opening (22), and the collecting structure (4) protrudes through the inner opening (22) for forming the interleaved arrangement.

6. The fluid filtering device as defined in claim 5, wherein the inner opening (22) of the adhesive (5) surrounds the collecting structure (4) and wherein the part of the collecting structure (4) being surrounded by the inner opening (22) of the adhesive (5) and the inner opening (22) of the adhesive (5) have corresponding shapes.

7. The fluid filtering device as defined in claim 6, wherein the collecting structure (4) comprises collecting channels (23), wherein longitudinal open sides of the collecting channels (23) face the filter (2) for collecting the filtered fluid from the filter (2), wherein the collecting channels (23) are arranged radially, wherein the shape of the inner opening (22) of the adhesive (5) corresponds to the radial arrangement of the collecting channels.

8. The fluid filtering device as defined in claim 1, the collecting structure (4) comprises collecting channels (23), wherein longitudinal open sides of the collecting channels (23) face the filter (2) for collecting the filtered fluid from the filter (2), wherein the fluid filtering device (1) comprises a channel structure (9) for guiding the fluid from the collecting structure (4) to a sensing region (8) for sensing the filtered fluid, wherein the substrate (3) comprises an opening (10) connecting the collecting structure (4) with the channel structure (9) for allowing the filtered fluid to flow from the collecting structure (4) to the channel structure (9), wherein the collecting channels (23) are arranged to guide the collected filtered fluid to the opening (10) of the substrate (3).

9. The fluid filtering device as defined in claim 9, wherein the collecting channels (23) are adapted such that at least one of the depth and the width of the collecting channels (23) decreases towards the opening (10).

10. The fluid filtering device as defined in claim 1, wherein the fluid filtering device (1) further comprises a channel structure (9) for guiding the fluid from the collecting structure (4) to a sensing region (8) for sensing the filtered fluid, wherein the substrate (3) comprises an opening (10) connecting the collecting structure (4) with the channel structure (9) for allowing the filtered fluid to flow from the collecting structure (4) to the channel structure (9), wherein the fluid filtering device (1) comprises a pin being located within the opening (10) for forming a circumferential shape of the opening (10).

11. The fluid filtering device as defined in claim 1, wherein the fluid filtering device (1) comprises a venting structure (24) for venting the collecting structure (4).

12. A sensing device for sensing a substance in a fluid, the sensing device (6) comprising:
a fluid filtering device (1) as defined in claim 1 for filtering the fluid,
a sensing region (8) being adapted for sensing the substance in the filtered fluid,
a channel structure (9) for guiding the filtered fluid from the filtering device (1) to the sensing region (8).

13. An analyzing device for analyzing a substance in a fluid, the analyzing device (14) being adapted to cooperate with a sensing device (6) as defined in claim 12 for analyzing the substance in the fluid, the analyzing device (14) comprising:
a sensing device (6) receiving section (15) for receiving the sensing device,
an analyzing unit (16) for analyzing the substance in the filtered fluid in the sensing region (8) of the sensing device, if the sensing device (6) is received in the sensing device (6) receiving section (15).

14. A fluid filtering method, wherein a fluid is filtered by a filter (2) and the filtered fluid is collected by a collecting structure (4), wherein the filter (2) is held in place with respect to the collecting structure (4) by an adhesive (5) and wherein the filter (2), the substrate (3) with the collecting structure (4) and the adhesive (5) are arranged such that the collecting structure (4) contacts the filter (2) and the filter (2) is attached to the substrate (3) by the adhesive (5).

15. A manufacturing method for manufacturing a fluid filtering device (1), the manufacturing method comprising following steps:
providing a filter (2) for filtering a fluid, a substrate (3) comprising a collecting structure (4) for collecting the filtered fluid from the filter (2), and an adhesive (5) for holding the filter (2) in place with respect to the collecting structure (4),
arranging the filter (2), the substrate (3) with the collecting structure (4) and the adhesive (5) such that the collecting structure (4) contacts the filter (2) and the filter (2) is attached to the substrate (3) by the adhesive (5).
